(19) United States
(12) Patent Application Publication
(10) Pub. No.: US 2013/0150659 A1
(43) Pub. Date: Jun. 13, 2013

(54) METHOD FOR AMELIORATING INSOMNIA

(71) Applicants: Fu-Zen Shaw, Tainan City (TW); Hsin-Hsin Yeh, Tainan City (TW)

(72) Inventors: Fu-Zen Shaw, Tainan City (TW); Hsin-Hsin Yeh, Tainan City (TW)

(73) Assignee: NATIONAL CHENG KUNG UNIVERSITY, Tainan City (TW)

(21) Appl. No.: 13/557,940

(22) Filed: Jul. 25, 2012

(30) Foreign Application Priority Data
Dec. 13, 2011 (TW) 100146061

(51) Int. Cl. A61M 21/02

(52) U.S. Cl. 600/28; 600/27

(57) ABSTRACT

A method for ameliorating an insomnia of a user having a brain wave includes steps of defining a specific frequency range of 8 to 12 Hz as a μ rhythm in the brain wave, detecting a total power under the specific frequency as a μ rhythm intensity of the user, providing one of an animation and a wave form representative of the μ rhythm intensity of the user, and inducing the μ rhythm of the user by watching one of the animation and the wave form by the user.

### CROSS REFERENCE TO RELATED APPLICATIONS

The application claims the benefit of Taiwan Patent Application No. 100146061, filed on Dec. 13, 2011, at the Taiwan Intellectual Property Office, the disclosures of which are incorporated herein in their entirety by reference.

### FIELD OF THE INVENTION

The present disclosure relates to a method for improving sleep quality and to assist falling asleep, especially to a method for ameliorating insomnia by using the μ rhythm

### BACKGROUND OF THE INVENTION

In the modern society, a lot of people have experiences of sleeplessness. However, the patients of chronic insomnia may seriously suffer in their daily lives. Currently, the frequently-used sleeping drugs, e.g. benzodiazepine, zolpidem, etc., are related to the facilitation of the retraining function of the nerve cells. In addition to drug therapies, neurofeedback treatments were proposed to solve the problem of insomnia.

Regarding the prior arts of the neurofeedback treatments, the earlier researches focus on a sensorimotor rhythm (SMR) at a frequency of 12 to 15 Hz and there was amelioration on the illnesses, e.g. epilepsy, obsessive-compulsive disorder (OCD), anger, anxiety, depression, migraine, etc. When a human being is relaxed, a specific SMR will surely appear in a somatosensory motor cortex area in the human brain. The specific SMR can be divided into two types based on the locations in the cortex area and the functions. The first type is a high frequency SMR of 15 to 20 Hz, is related to precentral sulcus motor cortex, and has the function associated with a start and an end of a motion. The second type is a low frequency SMR of 8 to 12 Hz (or called μ rhythm), is related to postcentral sulcus somatosensory cortex, and has the function associated with relaxation feeling and idling. Besides, the patents of U.S. Pat. No. 5,638,826 and U.S. Pat. No. 7,460,903 and the US patent applications of U.S. 20090306531 and U.S. 20090099627 did not disclose that the μ rhythm is applied to the neurofeedback training and is related to the amelioration of insomnia.

For overcoming the mentioned problems occurring in the prior art, a novel method for ameliorating insomnia is developed after a lot of researches, analyses and experiments by the inventors.

### SUMMARY OF THE INVENTION

In accordance with one aspect of the present disclosure, a method for ameliorating an insomnia of a user having a brain wave is provided. The method includes steps of defining a specific frequency range of 8 to 12 Hz as a μ rhythm in the brain wave, detecting a total power under the specific frequency as a μ rhythm intensity of the user, providing one of an animation and a wave form representative of the μ rhythm intensity of the user, and inducing the μ rhythm of the user by watching one of the animation and the wave form by the user.

In accordance with another aspect of the present disclosure, a method for ameliorating an insomnia of a user having a brain wave is provided. The method includes steps of defining a specific frequency range of 8 to 12 Hz as a μ rhythm in the brain wave, detecting a total power under the specific frequency range as a μ rhythm intensity of the user, providing a pattern showing the μ rhythm intensity of the user, and inducing the μ rhythm of the user by watching the pattern by the user.

In accordance with a further aspect of the present disclosure, a method for ameliorating an insomnia of a user having a brain wave is provided.

The method includes steps of providing a medium showing a μ rhythm intensity of the user, and perceiving the medium by the user for continuously causing the user to stay in one of states of emitting the μ rhythm and increasing the μ rhythm intensity.

The above objects and advantages of the present disclosure will become more readily apparent to those ordinarily skilled in the art after reviewing the following detailed descriptions and accompanying drawings, in which:

### BRIEF DESCRIPTION OF THE DRAWINGS

FIG. 1 is the schematic diagram showing a neurofeedback training device in one embodiment of the present disclosure;

FIG. 2 is the schematic diagram showing another neurofeedback training device in one embodiment of the present disclosure;

FIGS. 3(A) and 3(B) are the schematic diagrams showing an animation and a wave form in an interface for μ rhythm training in one embodiment of the present disclosure, respectively;

FIG. 4 is the schematic diagram showing relative power variations after neurofeedback training in one embodiment of the present disclosure;

FIG. 5 is the schematic diagram showing absolute power variations after neurofeedback training in one embodiment of the present disclosure;

FIGS. 6(A)-(D) are the schematic diagrams showing score variations on Pittsburg sleep quality inventory (PSQI), insomnia severity index (ISI), a somatic portion of the pre-sleep arousal scale (PSAS) and a cognitive portion of the PSAS in the embodiments of the present disclosure, respectively; and

FIG. 7(A)-(B) are the schematic diagrams showing variations on total sleep times and sleep efficiencies in the embodiments of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for the purposes of illustration and description only; it is not intended to be exhaustive or to be limited to the precise form disclosed.

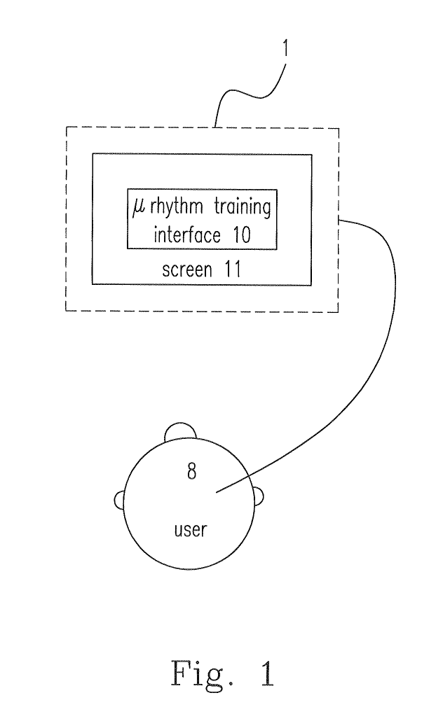

FIG. 1 shows a neurofeedback training device in one embodiment of the present disclosure. In FIG. 1, a neurofeedback training device 1 mainly includes a μ rhythm training interface 10 for enhancing the μ rhythm of the user 8 using the neurofeedback training device 1.

In the above embodiment, the μ rhythm training interface 10 can be a computer animation displayed on a display screen 11. During the training processes, the computer animation is working as an indicator indicating increment or decrement of the μ rhythm of the user 8. The user 8 can visually perceive the variation condition of the computer animation to operate a conditioning neurofeedback training so as to successfully induce the μ rhythm of the user 8 self.

In the above embodiment, enhancement of the μ rhythm of the user 8 includes the increment of the μ rhythm power, the increment of the appearance time period of the μ rhythm, or simultaneous increment of the both. In addition to the computer animation, other media can also be used in the present disclosure, e.g. a pattern, a diagram, a wave form, animation, sound, smell and temperature, as an indication of increment or decrement of the μ rhythm of the user 8.

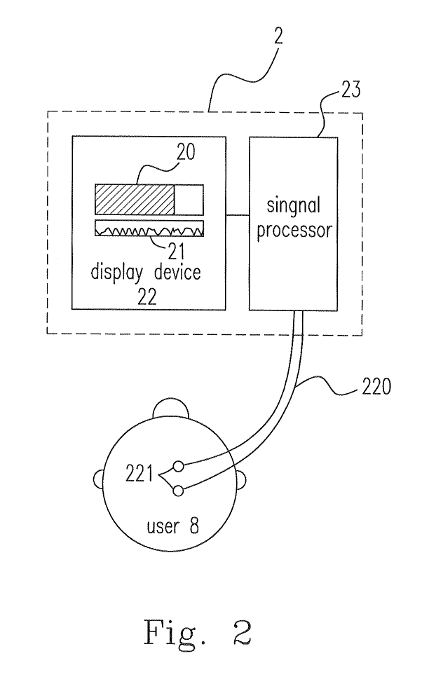

FIG. 2 shows another neurofeedback training device in one embodiment of the present disclosure. The neurofeedback training device 2 includes an animation 20, a wave form 21, a display device 22 and a signal processor 23, which receives and processes the signals related to the μ rhythm. The display device 22 is electrically connected to the signal processor 23, and displays the animation 20 and the wave form 21 thereon.

In the above embodiment, the signal processor 23 can be electroencephalograph, for example. This electroencephalograph can include sensor 220 connected with the brain of the user 8 for detecting the μ rhythm emitted from the brain of the user 8. The sensor 220 includes at least one pair of electrode patches 221. That is, the method of signal subtraction between the bipolar electrodes is adopted as the method for the signal capture to prevent undesired noise interference. Besides, the signal processor 23 receives the signals related to the μ rhythm at the parietal lobe area of the brain of the user 8, and the μ rhythm has a frequency of 8 to 12 Hz.

In the embodiment shown in FIG. 2, only one pair of electrode patches 221 are attached to the parietal lobe area of the brain of the user 8. However, in the practical applications, three pairs of electrode patches, for example, can be used to collect three sets of data and to obtain the average value for promoting the signal accuracy.

In the above embodiment, the display device 22 can be a computer monitor or a cellular phone screen, and the neurofeedback training device 2 can be a stationary device or a portable device. For example, the animation 20 or the wave form 21 can be installed in the cellular phone or the laptop computer, and the signal processor 23 can be connected to the these portable devices for the training by the user anyplace anytime.

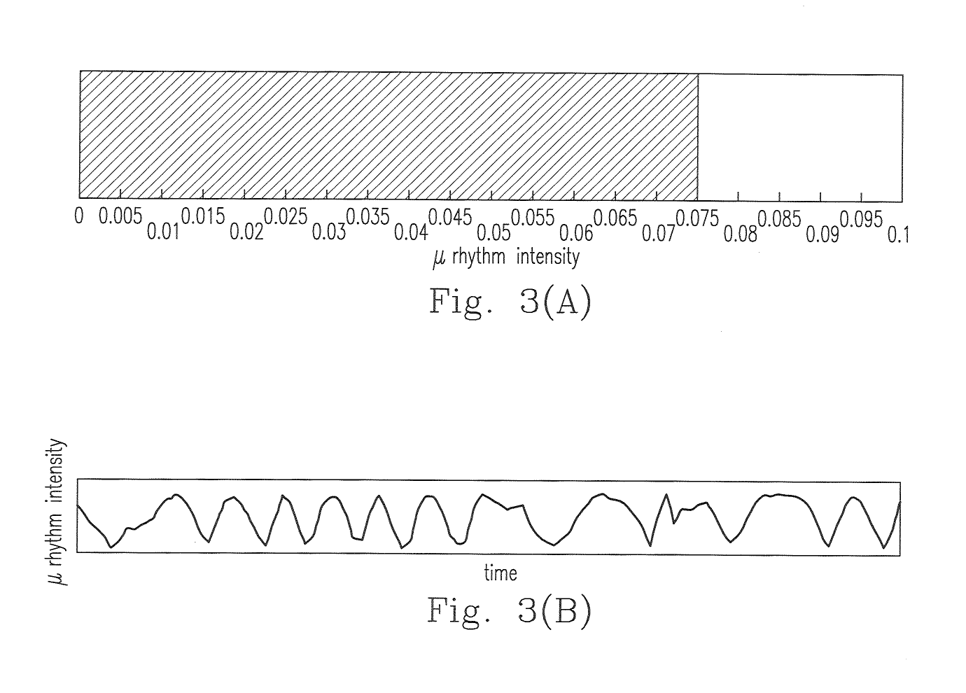

FIG. 3(A) shows the animation 20 of FIG. 2. In FIG. 3(A), the abscissa represents the intensity of the μ rhythm. The animation 20 includes a rightwards and leftwards movable latch. The more to the right the latch moves, the higher the intensity of the μ rhythm emitted from the user 8. FIG. 3(B) shows the wave form 21 in FIG. 2. In FIG. 3(B), the abscissa represents time, the wave peak indicates high intensity of the μ rhythm, and the wave valley indicates low intensity of the μ rhythm. FIG. 3(B) shows that the stable plateau lasts longer as the training time become longer.

The detailed experimental methods and diagrams are described and shown below for proving the efficacies of ameliorating insomnia by applying the neurofeedback training systems and methods in the present disclosure.

### Subjects:

All the voluntary insomnia patents were interviewed before participating the experiments. After the completion of the analysis tools, e.g. Pittsburg sleep quality inventory and insomnia severity index, and the questionnaire, e.g. personal decease history and living habit inspection, some subjects meeting the requirements were primarily sieved out. Each subject under the experiments was requested to wear an actiwatch for recording the activities when going to sleep at home, and to record the daily sleep conditions into a sleep diary, and finally was arranged to go through a polysomnography (PSG) in the laboratory for inspecting the insomnia conditions. The selected voluntary subjects must meet the following requirements: (1) age in a range of 18 to 60 years old; (2) sleeplessness occurring three times or more in a week for one month; (3) tiredness, somnolence, disturbance, distraction or physical discomfort appearing in day time with negative influences on learning or working; (4) falling asleep more than 30 minutes after going to bed or waking up longer than 30 minutes; and (5) sleep efficiency (=actual sleep time/lying time on the bed×100%) not higher than 85%, but could not have the following symptoms: (1) neuropathy or mentality related deceases, e.g. psychosis, major depressive disorder, mental retardation, dementias, drug abuse, etc.; (2) deceases liable to affect sleep, e.g. cancer, pain decease, cardiovascular disease, sleep apnea, restless legs syndrome, periodic limb movements, etc.; (3) sleeplessness in specific conditions, e.g. pregnancy, working in a night shift or irregular sleep habit. After the subjective tools and questionnaire and objective sleep inspection, the subjects meeting all the requirements were randomly assigned to a control group of a random neurofeedback and an experimental group of the μ rhythm neurofeedback.

After the analysis of the questionnaire results, total 36 subjects with self-recognized long-term sleeplessness were selected, and then after the objective inspection of the sleep conditions by wearing the actiwatches, total 14 subjects met the requirements and randomly assigned to the random neurofeedback group and the μ rhythm neurofeedback group. There was no difference in age, sex and years of insomnia between the two groups of subjects.

### Pre-Sleep Arousal Scale, PSAS

The PSAS is constructed based on the clinical observation and pre-sleep experiences described by insomnia patients for assaying pre-sleep somatic and cognitive scales. The PSAS contains 16 questions, and is divided into tow types of arousal scales, wherein the somatic scale contains the first to eighth questions reflecting the pre-sleep physiological conditions, e.g. heart beats, muscle tone, ecphydesis, etc.; while the cognitive scale contains the ninth to sixteenth questions regarding pre-sleep worry, psychological alertness, non-stoppable thinking, etc. Five scales are adopted in the questionnaire, wherein 1 represents no feeling, and 5 represent extremely strong feeling. The scores of the somatic scale and the cognitive scale can be obtained from the answers to the questionnaire. The higher score, the higher the somatic or cognitive scale.

### Pittsburg Sleep Quality Inventory, PSQI

The PSQI is used to assay the sleep quality in the past one month, and contains total nineteen items with seven major indications, including subjective sleep quality, sleep onset latency, total sleep time, habitual sleep efficiency, sleep disturbance, use of sleeping medication, daytime dysfunction, etc. The score in each aspect is calculated and then the total scope is summed up. The total score is in a range of 0 to 21 points. The higher the score, the poorer the sleep quality. There is a division for every five points for distinguishing poor and good sleep quality. The total score higher than 5 represents poor sleep quality. The detailed scoring method is depicted below.

The subjective sleep quality is assayed by the score of the ninth question alone to allow the subject to self-evaluate the subjective sleep quality in the past one month. The scoring method is designed as follows. 0 point: excellent; 1 point: fair; 2 points: poor; and 3 points: bad. The higher score, the more dissatisfied with the sleep quality by the subject self.

The sleep onset latency is assayed by the scores of the second and the 5a^{th} questions. The second question is asked about how much time is spent for the subject to fall asleep after going to bed. The scoring is based on the spent time. The scoring method is designed as follows. 0 point: less than 5 minutes; 1 point: 16 to 30 minutes; 2 points: 31 to 60 minutes; and 3 points: longer than 60 minutes. The 5a^{th} question is asked about the frequency at which the subject can not fall asleep in 30 minutes after going to bed. The scoring method is designed as follows. 0 point: never happening; 1 point: less than once a week; 2 points: once to twice a week; and 3 points: three times a week or more. After the assays of the second and 5a^{th} questions, the scores of these two questions are summed up as the total score of the sleep onset latency. The scoring method is designed as follows. 0 point: when the total score is 0 point; 1 point: when the total score is 1-2 point; 2 points: when the total score is 3-4 point; and 3 points: when the total score is 5-6 point. The higher score, the longer sleep onset latency.

The total sleep time is obtained from the fourth question alone based on the sleep time subjectively estimated by the subject. The scoring method is designed as follows. 0 point: longer than 7 hours; 1 point: longer than or equal to six hours but less than 7 hours; 2 points: longer than or equal to 5 hours but less than six hours; and 3 points: less than 5 hours. The higher score, the less sleep time.

The habitual sleep efficiency is a percentage obtained by dividing the total sleep time (the fourth question) by the time spent on lying on the bed (subtracting the first question from the third question). The time point of going to bed, the time point of falling asleep, the time point of waking up in the morning, and real time of falling asleep are answered in the first to fourth questions, respectively. The scoring method is designed as follows. 0 point: higher than or equal to 85%; 1 point: 75-84%; 2 points: 65-74%; and 3 points: lower than 65%. The higher score, the lower habitual sleep efficiency.

The sleep disturbance is obtained by the total scores of the 5b^{th} to 5i^{th} questions regarding the occurrence frequency of the sleep disturbance suffered by the subject. The scoring method for each of the 5b^{th} to 5i^{th} questions is designed as follows. 0 point: never happening; 1 point: less than once a week; 2 points: once to twice a week; and 3 points: three times a week or more. Then the scores of the 5b^{th} to 5i^{th} questions are summed up to obtain the total score. The scoring method for the total score is designed as follows. 0 point: when the total score is 0 point; 1 point: when the total score is 1-9 points; 2 points: when the total score is 10-18 points; and 3 points: when the total score is 19-27 points. The higher score, the more serious sleep disturbance.

The use of sleeping medication is assayed based on the sixth question regarding the frequency of taking sleep medicines by the subject.

The scoring method is designed as follows. 0 point: never using; 1 point: less than once a week; 2 points: once or twice a week; and 3 points: three times a week more. The higher score, the more frequently using the sleep medicines.

The daytime dysfunction is assayed by summing up the scores of the seventh and eighth questions. The seventh question is used to assay the frequency of being unable to keep sober when being engaged in activities in day time. The scoring method is designed as follows. 0 point: never happening; 1 point: less than once a week; 2 points: once or twice a week; and 3 points: three times a week or more. The eighth question is used to assay the torment level when the user has to pluck up to finish jobs. The scoring method is designed as follows. 0 point: free from torment; 1 point: little torment; 2 points: somewhat torment; and 3 points: serious torment. Then the scores of the seventh and eighth questions are summed up to obtain the total score. The scoring method for the total score is designed as follows. 0 point: when the total score is 0 point; 1 point: when the total score is 1-2 points; 2 points: when the total score is 3-4 points; and 3 points: when the total score is 5-6 points. The higher score, the more serious the daytime dysfunction.

### Insomnia Severity Index (ISI)

The ISI is used to understand the perception level on the sleeplessness issue by the subject and to assay the insomnia severity of the subject as a whole. The questionnaire consists of sevens questions. The first three questions are used to assay the of the subjective insomnia issue, including three types: difficulty of falling asleep, difficulty of staying asleep and waking up too early. The fourth to seventh questions are used to assay sleep satisfaction level, influence levels on the subject by self-perception and the perception by others, and attention level on the insomnia In this experiment, the index is divided into two items, insomnia severity and insomnia torment. The insomnia severity is assayed by summing up the scores of the first three questions. The insomnia torment is assayed by obtaining the total scores of ISI. Five scales are adopted in the questionnaire. The appropriate points are selected by the subject based on the subject self's condition. The points of all the questions are accumulated as a total score in a range of 0 to 28 points, where 0-7 points: no clinically significant insomnia; 8-14 points: sub-threshold insomnia; 15-21 points: moderate insomnia; and 22-28 points: severe insomnia The ISI is highly related to sleep quality.

### Neurofeedback Training System

The electrode cap from Neuroscan corporation was adopted for the measurement of brain waves, and was coupled to a laptop computer and a four-channel signal amplifier, which was developed in the inventor's laboratory and included three electroencephalogram (EEG) amplifiers and one electrocardiogram (ECG) for recording the physiological signals of brain waves and heart beats. The amplified signal was converted into digital signals through the analog/digital signal converter set DAQ-6024E and CB-68LP from National Instrument Corporation. The signals of the brain wave for each second were captured and processed through fast Fourier transform (FFT) to convert the voltage variations from the brain waves into power variation on frequency spectra. Then the total powers in the corresponding frequency bands, i.e. 8-12 Hz for the μ rhythm group and 7-20 Hz for randomness group, for the two groups were calculated and instantly shown on the computer screen in red histograms in combination with animated pictures to feedback the instant brain wave powers to the subjects in the neurofeedback experiments.

### Polysomnography

The Siesta 80 wireless ambulatory recorder was used to record video-EEG data during the sleep in the laboratory, wherein the records included seven EEGs, two electromyograms (EMGs) and two electrooculograms (EOGs). The frequency bandwidth for EEGs was set at 0.15-100 Hz, and the frequency bandwidths for EMGs and EOGs were set at 1-100 Hz. The measurement locations of EEG electrodes were Fz, C3, C4, F3, F4, Al and A2, and were disposed according to the international standard system. The EMG electrodes were attached to the locations about 2 cm below the two ends of the mouth. The EOG electrodes were attached to the locations about 1 cm above the end of left eye and about 1 cm below the end of right eye. All the signals were collected at a frequency of 500 Hz.

### Actiwatch

The actiwatches were cooperatively developed by the inventor's laboratory, Institute of Computer Science and Information Engineering, National Cheng Kung University in Taiwan and Delta Electronics. The actiwatches could be carried by the subjects to their homes to record their sleep conditions due to the slim design of the actiwatches. The prototype of the actiwatch was developed and tested, and the tested results were compared with those by the polysomnography. The signals appearing in the actiwatch during the sleep process were confirmed with the relevance to the activities of arousals and body turns, and had the accuracy higher than 95%.

### Experimental Procedures

The original signals were instantly converted into frequency power through frequency spectrum analysis for neurofeedback training. The frequency bands of the feedback signals were divided into tow groups, i.e. the μ rhythm group with the frequency of 8-12 Hz and the random signal group with the frequency of 7-20 Hz. The feedback signals in the μ rhythm group were limited to the frequency band of 8-12 Hz; while four bands, e.g. 11-15, 16-20, 15-19 and 7-10 Hz, were randomly selected and used for the feedback signals in the random signal group. The subjects were randomly assigned to one of the two groups for power frequency band training. The processes of the experiments could be divided into three major processes: pre-test of neurofeedback training, one-month neurofeedback training and post-test of neurofeedback training.

The sleep assays and sleep conditions were recorded for the subjects in the pre-test of neurofeedback training and the post-test of neurofeedback training. The sleep assays includes Pittsburg sleep quality inventory, insomnia severity index and pre-sleep arousal scale. The Siesta 80 wireless ambulatory recorder was used for sleep recording and detailed sleep parameter analysis. In order to obtain more objective sleep related data, the actiwatch was used to record the objective actigraphy during the sleep process of the subject in each day in the processes of the pre-test of neurofeedback training, the one-month neurofeedback training and the post-test of neurofeedback training.

### Sleep Analysis

The sleep brain waves in the whole night recorded by the PSG were primarily analyzed by using computer automatic sleep diagnostic analysis. Then the sleep conditions were manually corrected according to R&K principle (Rechtschaffen and Kales, 1968). The sleep onset latency (SOL), wake after sleep onset (WASO), sleep efficiency, total sleep time (TST), time in each period of sleep, the number and density of the second sleep spindle were analyzed. The method of two-way repeat measure analysis of variance (ANOVA) was used to check if there is difference in each parameter before and after the trainings by comparing each parameter for the experimental group and the control group.

The actiwatches recorded sleep variations in one month (12 records). The analysis software was used to analyze the SOL, WASO, sleep efficiency and TST. The method of two-way repeat measure ANOVA was used to check if there is difference in each parameter before and after the trainings by comparing each parameter for the experimental group and the control group.

### Questionnaire Analysis

The method of two-way repeat measure ANOVA was used to check if there is difference in the scores of the Pittsburg sleep quality inventory, insomnia severity index and pre-sleep arousal scale between the experimental group and the control group before and after the neurofeedback training.

### Analysis Results of Brain Wave Signals

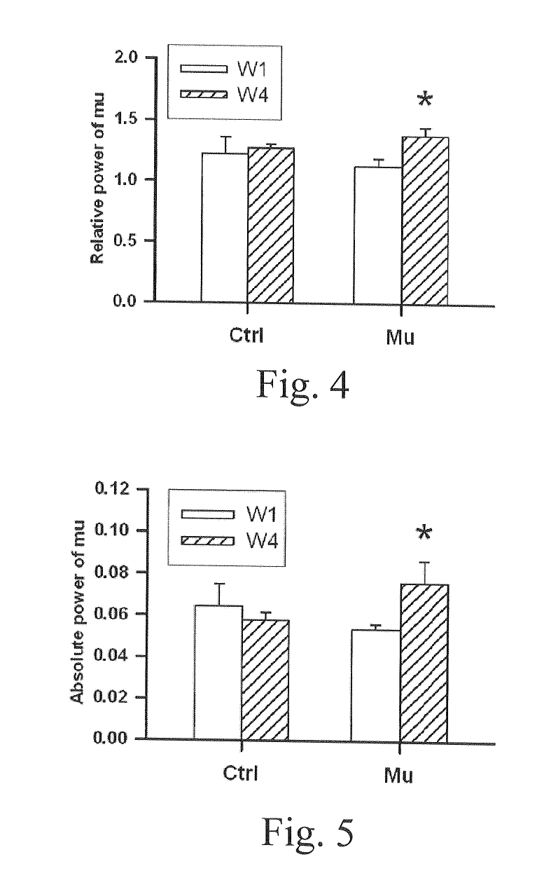

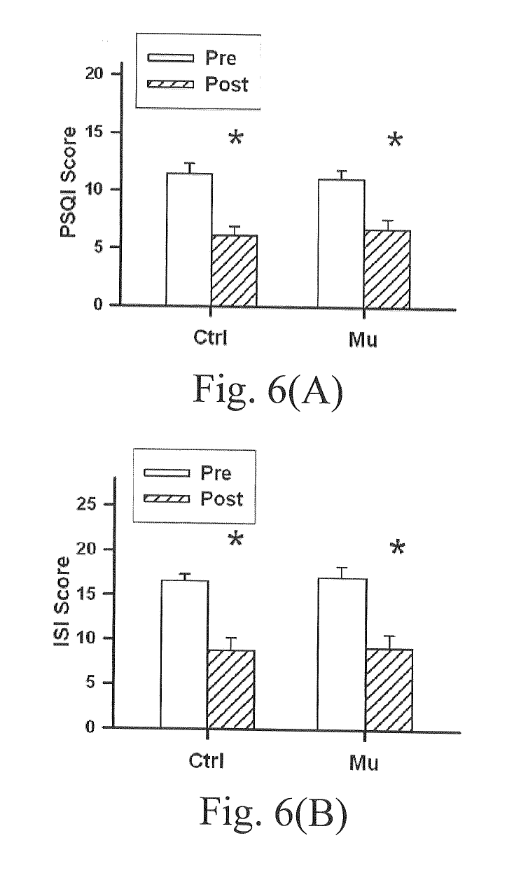

FIG. 4 shows relative power variations for the control group and the μ rhythm group after the neurofeedback training. FIG. 5 shows absolute power variations for the control group and the μ rhythm group after the neurofeedback training. The blank column in the histogram indicates the result for the first week, and the shaded column in histogram indicates that for the fourth week. The power variations of the first and fourth weeks were analyzed by using two-way repeat measure. The relative and absolute powers for the μ rhythm group were significantly enhanced after the neurofeedback training (p<0.05).

### Analysis Results of Subjective Sleep Parameters

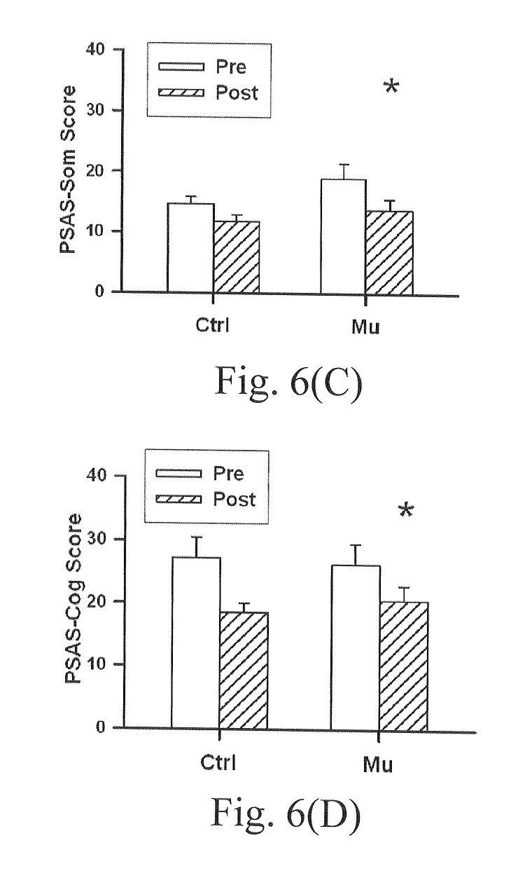

After the analysis of each parameter by using two-way repeat measure ANOVA, the scores of the Pittsburg sleep quality inventory for both the control group and the μ rhythm group after the neurofeedback training were apparently reduced (F=87.797, p<0.001) as shown in FIG. 6(A), the scores of the insomnia severity scale for both the control group and the μ rhythm group after the neurofeedback training were also apparently reduced (F=41.389, p<0.001) as shown in FIG. 6(B), the score of the physiological portion of the pre-sleep arousal scale for the μ rhythm group was apparently reduced (t=3.693, p<0.05) but not for the control group as shown in FIG. 6(C), and the scores of the cognitive portion of the pre-sleep arousal scale for both the control group and the μ rhythm group after the neurofeedback training were apparently reduced (F=27.210, p<0.001) as shown in FIG. 6(D).

### Analysis Results of Objective Sleep Parameters

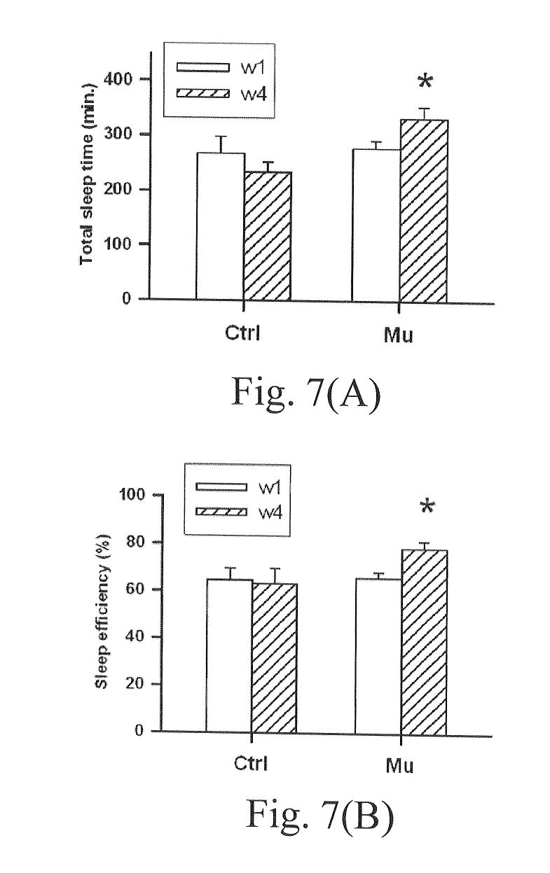

The actiwatches were used to record sleep variations of the subjects through the neurofeedback training. Each parameter for the first and fourth weeks was compared by using two-way repeat measure ANOVA. The total sleep time for the μ rhythm group was significantly elongated after the training (t=2.370, p<0.05) as shown in FIG. 7(A). In addition, the sleep efficiency for the μ rhythm group after the training was also significantly promoted (t=5.904, p<0.001) as shown in FIG. 7(B).

### Efficacies of Ameliorating Insomnia in the Present Disclosure

After the μ rhythm neurofeedback training, not only the results in the subjective sleep questionnaires clinically used, such as Pittsburg sleep quality inventory, insomnia severity index and pre-sleep arousal scale, showed significant efficacies of ameliorating insomnia, but also the total sleep time recorded by the actiwatch and the sleep efficiency after one-month training were significantly elongated and promoted, respectively.

Some embodiments of the present disclosure are described in the followings.

1. A method for ameliorating an insomnia of a user having a brain wave, comprising steps of defining a specific frequency range of 8 to 12 Hz as a μ rhythm in the brain wave, detecting a total power under the specific frequency as a μ rhythm intensity of the user, providing one of an animation and a wave form representative of the μ rhythm intensity of the user, and inducing the μ rhythm of the user by watching one of the animation and the wave form by the user.

2. A method for ameliorating an insomnia of a user having a brain wave, comprising steps of defining a specific frequency range of 8 to 12 Hz as a μ rhythm in the brain wave, detecting a total power under the specific frequency range as a μ rhythm intensity of the user, providing a pattern showing the μ rhythm intensity of the user, and inducing the μ rhythm of the user by watching the pattern by the user.

3. A method for ameliorating an insomnia of a user having a brain wave including a μ rhythm, comprising steps of providing a medium showing a μ rhythm intensity of the user, and perceiving the medium by the user for continuously causing the user to stay in one of states of emitting the μ rhythm and increasing the μ rhythm intensity.

4. A method of any one of the preceding embodiments, wherein the medium includes one selected from a group consisting of a pattern, a wave form, an animation, a sound, a smell and a temperature.

5. A method of any one of the preceding embodiments, for enhancing a subjective sleep quality of the user.

6. A method of any one of the preceding embodiments, for enhancing an objective sleep quality of the user.

7. A method of any one of the preceding embodiments, further comprising before the perceiving step a step of recognizing step by which the user recognizes what kind of mental action enables the user to stay in the one state.

8. A method of any one of the preceding embodiments, further comprising a step of installing the medium in a stationary device.

9. A method of any one of the preceding embodiments, further comprising a step of installing the medium in a portable device.

10. A method of any one of the preceding embodiments, further comprising a step of repeating a training including the steps in any one of the embodiments 1-3 three times a week.

11. A method of any one of the preceding embodiments, wherein the μ rhythm has a specific frequency of 8 to 12 Hz.

While the disclosure has been described in terms of what is presently considered to be the most practical and preferred embodiments, it is to be understood that the disclosure needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for ameliorating an insomnia of a user having a brain wave, comprising steps of:
defining a specific frequency range of 8 to 12 Hz as a μ rhythm in the brain wave;
detecting a total power under the specific frequency as a μ rhythm intensity of the user;
providing one of an animation and a wave form representative of the μ rhythm intensity of the user; and
inducing the μ rhythm of the user by watching one of the animation and the wave form by the user.

2. A method of claim 1, for enhancing a subjective sleep quality of the user.

3. A method of claim 1, for enhancing an objective sleep quality of the user.

4. A method of claim 1, further comprising a step of installing one of the animation and the wave form in a stationary device.

5. A method of claim 1, further comprising a step of installing one of the animation and the wave form in a portable device.

6. A method of claim 1, further comprising a step of repeating a training including the steps in claim 1 three times a week.

7. A method for ameliorating an insomnia of a user having a brain wave, comprising steps of:
defining a specific frequency range of 8 to 12 Hz as a μ rhythm in the brain wave;
detecting a total power under the specific frequency range as a μ rhythm intensity of the user;
providing a pattern showing the μ rhythm intensity of the user; and
inducing the μ rhythm of the user by watching the pattern by the user.

8. A method of claim 7, for enhancing a subjective sleep quality of the user.

9. A method of claim 7, for enhancing an objective sleep quality of the user.

10. A method of claim 7, further comprising a step of installing the pattern in a stationary device.

11. A method of claim 7, further comprising a step of installing the pattern in a portable device.

12. A method of claim 7, further comprising a step of repeating a training including the steps in claim 7 three times a week.

13. A method for ameliorating an insomnia of a user having a brain wave including a μ rhythm, comprising steps of:
providing a medium showing a μ rhythm intensity of the user; and
perceiving the medium by the user for continuously causing the user to stay in one of states of emitting the μ rhythm and increasing the μ rhythm intensity.

14. A method of claim 13, wherein the medium includes one selected from a group consisting of a pattern, a wave form, an animation, a sound, a smell and a temperature.

15. A method of claim 13, for enhancing one of a subjective sleep quality and an objective sleep quality of the user.

16. A method of claim 13, further comprising before the perceiving step a step of recognizing step by which the user recognizes what kind of mental action enables the user to stay in the one state.

17. A method of claim 13, further comprising a step of installing the medium in a stationary device.

18. A method of claim 13, further comprising a step of installing the medium in a portable device.

19. A method of claim 13, further comprising a step of repeating a training including the steps in claim 13 three times a week.

20. A method of claim 13, wherein the μ rhythm has a specific frequency of 8 to 12 Hz.
